(19) United States
(12) Patent Application Publication
(10) Pub. No.: US 2026/0007253 A1
(43) Pub. Date: Jan. 8, 2026

(54) DISPOSABLE DIAPER CHANGING KIT

(71) Applicant: Natalie Pearson, Chanhassen, MN (US)

(72) Inventor: Natalie Pearson, Chanhassen, MN (US)

(21) Appl. No.: 18/762,312

(22) Filed: Jul. 2, 2024

(51) Int. Cl. A47D 15/00; A61F 13/551

(52) U.S. Cl. CPC A47D 15/003; A61F 13/55115; A61F 13/5512; A61F 13/5519

(57) ABSTRACT

A disposable diaper changing kit includes a container and diaper changing materials. The container includes a base panel and side panels connected thereto. The container is expandable from a collapsed position in which the side panels and the base panel are folded over and an expanded position in which the side panels are transverse to the base panel and together define a free-standing receptacle with an open end to receive used or soiled items dropped therein. The diaper changing materials include a disposable diaper and disposable wipes, wherein the diaper changing materials are stored within the container when the container is in the collapsed position.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to kits and accessories for the care of children and more particularly pertains to a new disposable diaper changing kit for providing a caregiver with materials to change a child, which kit includes a container that functions as both the storage container for the diaper changing materials and as a disposable free-standing receptacle to provide a place to drop used or soiled items during or after the changing and cleaning process. It should be noted that the terms “child” or “children” as used herein refer to any child of any age who may wear a disposable diaper or similar protective or absorbent garment, including babies, infants, newborns, toddlers, younger children, and older children.

### (2) Description of Related Art Including Information Disclosed Under 37 CFR 1.97 and 1.98

The prior art relates to kits and accessories for the care of children. The prior art, as best understood, does not disclose a disposable diaper changing kit includes a container and diaper changing materials, wherein the container stores the diaper changing materials prior to use and can be expanded into a free-standing receptacle to receive used or soiled items dropped therein.

### BRIEF SUMMARY OF THE INVENTION

An embodiment of the disclosure meets the needs presented above in a disposable diaper changing kit generally comprising a container and diaper changing materials. The container includes a base panel and side panels connected thereto. The container is expandable from a collapsed position in which the side panels and the base panel are folded over and an expanded position in which the side panels are transverse to the base panel and together define a free-standing receptacle with an open end to receive used or soiled items dropped therein. The diaper changing materials include a disposable diaper and disposable wipes, wherein the diaper changing materials are stored within the container when the container is in the collapsed position.

The disposable diaper changing kit allows a user to conveniently transport diaper changing materials in a closed container, but also provides the user with a disposable free-standing receptacle, much like a wastebasket or bin, that may be placed close to the user during the changing and cleaning process when the user's hands are occupied with changing and controlling the child. Since the receptacle is free-standing, the user can then drop soiled or used items, such as a soiled diaper, used disposable wipes, and other such items, into the free-standing receptacle with one hand while keeping the other hand free for other tasks in the cleaning and changing process. In addition, the user does not need to substantially manipulate the soiled or used items during the disposal process, thereby minimizing additional soiling or contamination of the user's hands.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a disposable diaper changing kit according to an embodiment of the disclosure.

FIG. 2 is a top perspective view of an embodiment of the disclosure.

FIG. 3 is a front view of a first alternative embodiment of the disclosure.

FIG. 4 is a perspective view of the first alternative embodiment of the disclosure.

FIG. 5 is a perspective view of the first alternative embodiment of the disclosure.

FIG. 6 is a perspective view of the first alternative embodiment of the disclosure.

FIG. 7 is a top perspective view of a second alternative embodiment of the disclosure.

FIG. 8 is a top perspective view of the second alternative embodiment of the disclosure.

FIG. 9 is a top perspective view of the second alternative embodiment of the disclosure.

FIG. 10 is a top perspective view of the second alternative embodiment of the disclosure.

### DETAILED DESCRIPTION OF THE INVENTION

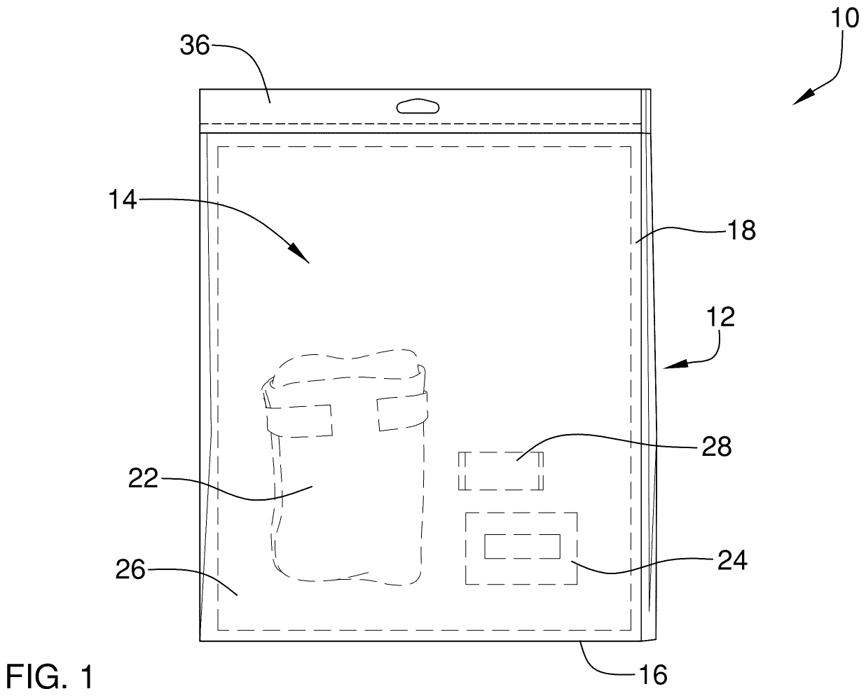

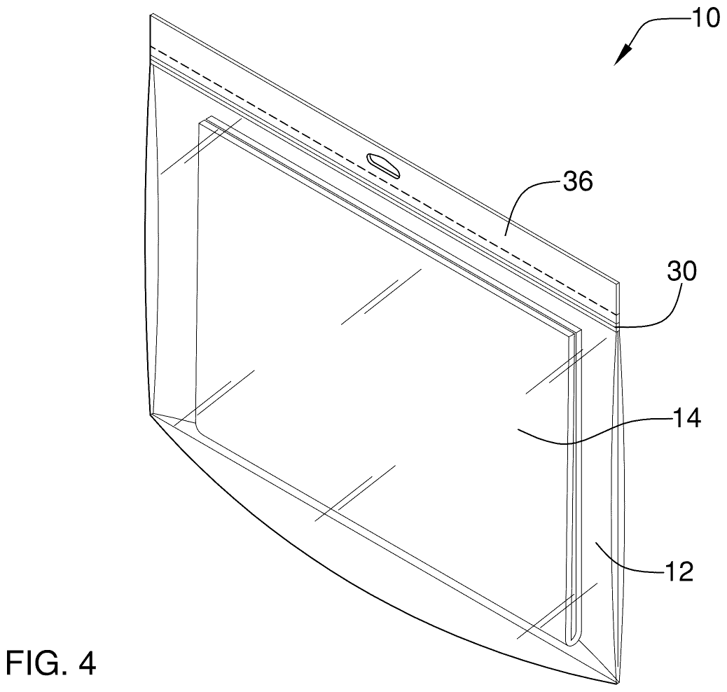

With reference now to the drawings, and in particular to FIGS. 1 through 10 thereof, a new disposable diaper changing kit embodying the principles and concepts of an embodiment of the disclosure and generally designated by the reference numeral 10 will be described.

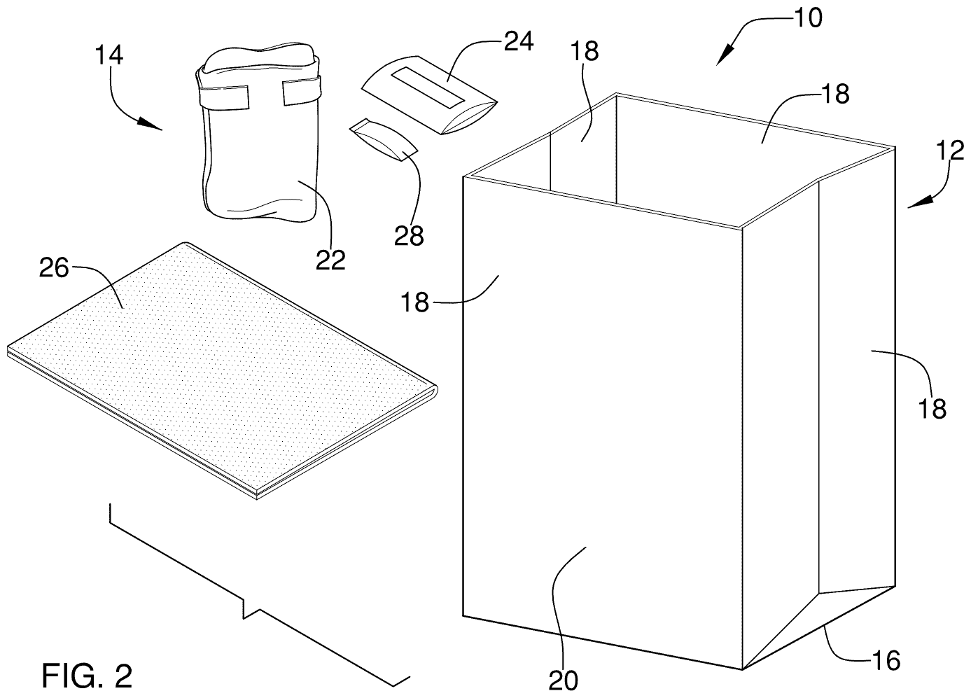

As best illustrated in FIGS. 1 through 10, the disposable diaper changing kit 10 generally includes a container 12 and diaper changing materials 14. As shown in FIG. 2, the container 12 includes a base panel 16 and side panels 18 connected thereto. The container 12 is expandable from a collapsed position, as shown in FIG. 1, in which the side panels 18 and the base panel 16 are folded over and an expanded position, as shown in FIG. 2, in which the side panels 18 are transverse to the base panel 16 and together define a free-standing receptacle 20 with an open end to receive used or soiled items dropped therein. The diaper changing materials 14 include a disposable diaper 22 and disposable wipes 24, wherein the diaper changing materials 14 are stored within the container 12 when the container 12 is in the collapsed position.

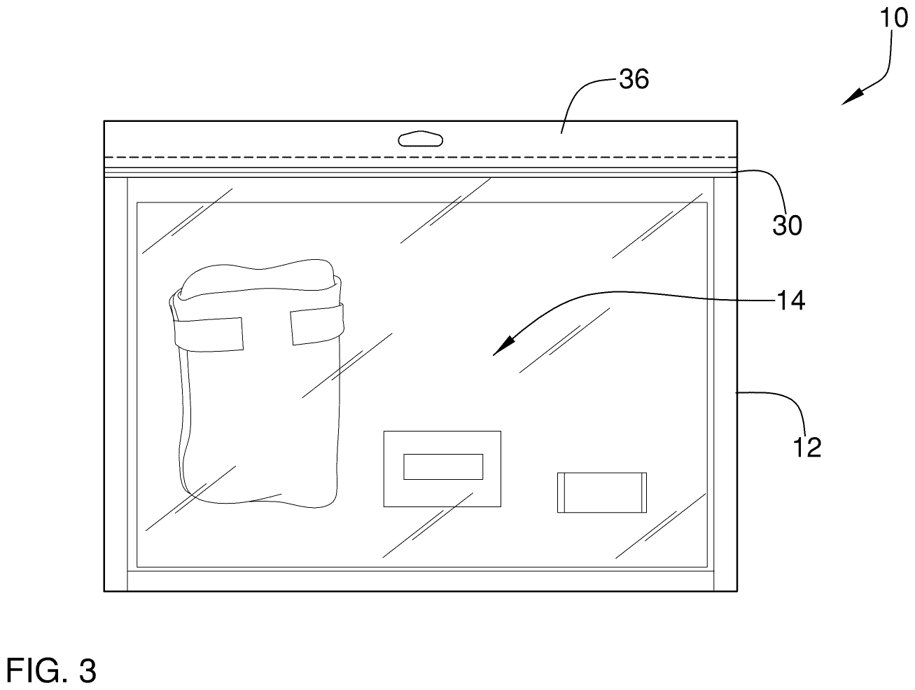

In one possible embodiment, the diaper changing materials 14 also include a disposable changing mat 26, and also could include a disposable hand sanitizing packet 28, as shown in FIGS. 2 and 3. The hand sanitizing packet 28 could contain hand sanitizing liquid or gel or hand sanitizing wipes. In at least one possible embodiment, the diaper changing materials 14 could include other disposable items relating to cleaning and changing of a child, such as powders, sprays, lotions, and creams.

As shown in FIG. 1, the container 12 includes a tear-off cover 36 to seal the open end of the container 12 prior to use. As shown in FIG. 2, the side panels 18 have a height substantially greater than a width of the base panel 16, such that the container 12 is shaped much like a gift bag or similar structure.

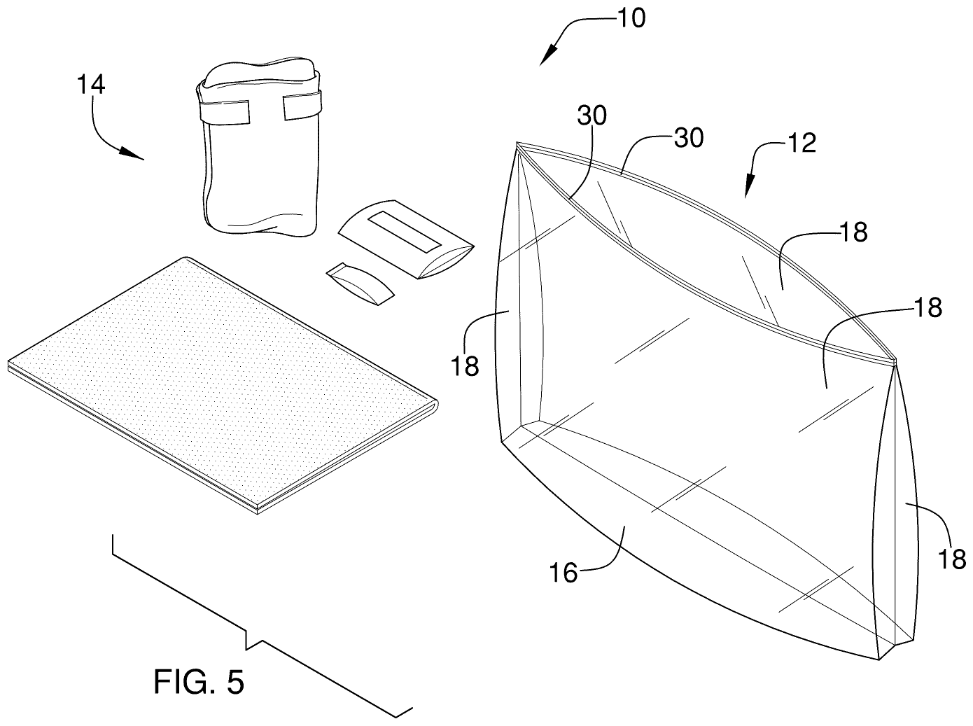

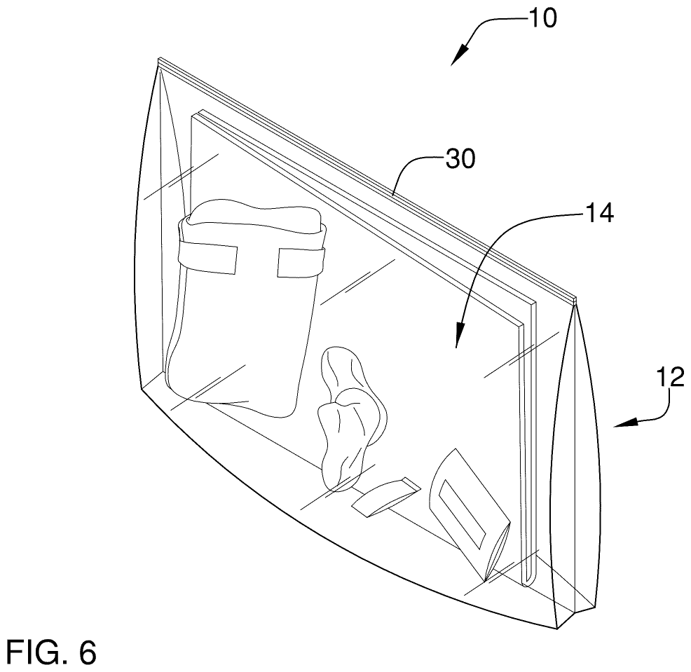

Regardless of the exact dimensions and design, the container 12, when in its open state, should be able to stand on its own to provide the user with a free-standing receptacle 20. In that respect, the base panel 16 and the side panels 18 are made of a semi-rigid material, such as a card stock or heavier weight paper or cardboard material, or possibly a plastic material or plastic-coated material, which can be resiliently flexible or foldable. The material could be fully or at least partially transparent. Alternatively, the container 12 could have flexible or foldable panels 16, 18 supported by a foldable frame, such as a wire frame or similar. Therefore, the container 12 can have various configurations not limited to the design shown in the figures. In that regard, FIGS. 3 through 6 show a first alternative embodiment of the disposable diaper changing kit 10. This design is made of a semi-rigid plastic that can be expanded slightly and has more of an envelope or pouch design. The container 12 in this case includes a sealing structure 30 designed to seal the open end of the container 12 after used or soiled items are dropped into the container 12. The sealing structure 30 can be a pair of sealing lips like a plastic storage or freezer bag, which can be opened as shown in FIG. 5, then re-sealed after use as shown in FIG. 6 to contain odors and maintain hygienic conditions.

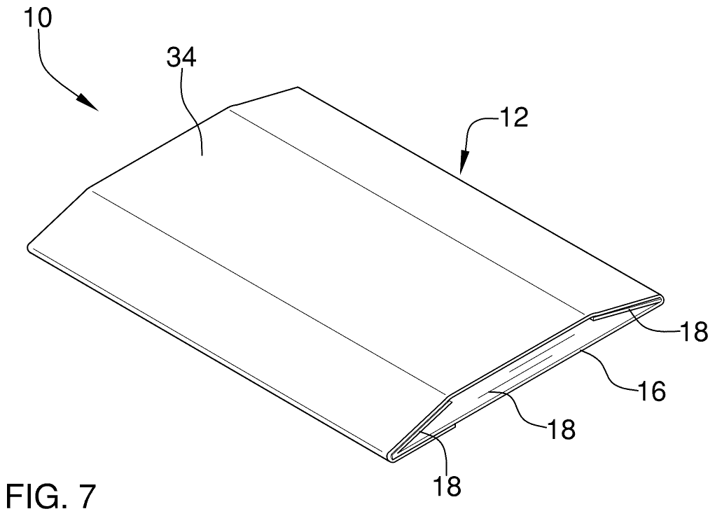

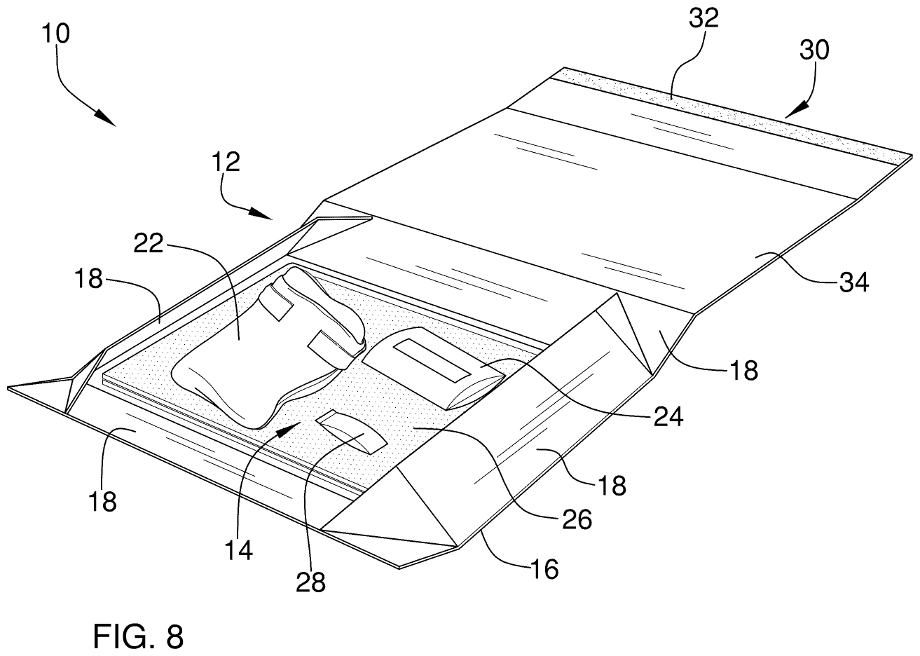

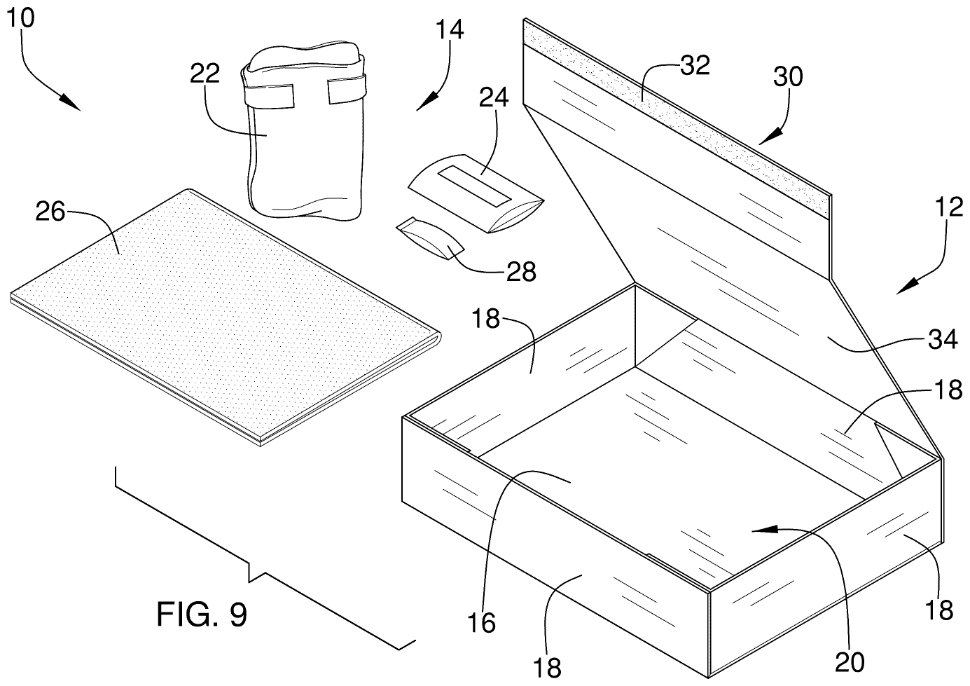

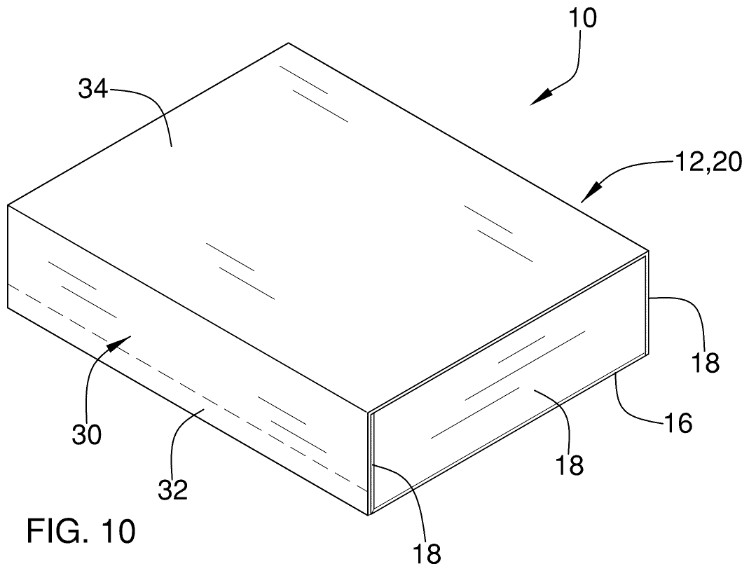

FIGS. 7 through 10 show a second alternative embodiment of the disposable diaper changing kit 10. The side panels 18 are movable between a folded position over the base panel 16, as shown in FIG. 7, and an unfolded position transverse to the base panel 16, as shown in FIGS. 9 and 10, to change the container 12 from a closed state to an open state in which the base panel 16 and the side walls define a free-standing receptacle 20, as shown in FIG. 10, with an open end to receive used or soiled items dropped therein. The diaper changing materials 14 include a disposable diaper 22 and disposable wipes 24, wherein the diaper changing materials 14 are stored within the container 12 when the container 12 is in the collapsed position, as shown in FIG. 8. In this embodiment, the sealing structure 30 could be in the form of an adhesive strip 32. While not shown, the embodiment in FIGS. 1 and 2 could include a sealing structure 30 of any type disclosed herein.

In this second alternative embodiment, the container 12 includes a cover panel 34 pivotably connected to one of the side panels 18. The cover panel 34 is unfoldable to an upwardly angled or vertical open position, as shown in FIG. 9, and foldable over the open end of the container 12 to close said container 12 when in the open state and containing used or soiled items, as shown in FIG. 10. The cover panel 34 is designed to stay in this position so the user does not have to hold the container 12 open while performing cleaning and changing of a child. The sealing structure 30 can be located on the cover panel 34. This design allows closure of the container 12 without the need or risk of touching the interior of the container 12 or the soiled contents therein, thereby allowing the user to avoid further contamination of the user's hands. In this second alternative embodiment, the side panels 18 have a height substantially less than the width thereof, such that the free-standing receptacle 20 is in the form of a low-profile box.

In use, a user intending to change a child having a soiled diaper takes the disposable diaper changing kit 10 and expands the container 12 from the collapsed position to form the free-standing receptacle 20. The user removes the disposable diaper 22 and the disposable wipes 24 from the container 12, as well as any other items included in the disposable diaper changing kit 10, such as the disposable changing mat 26 and the hand sanitizing packet 28, either prior to fully opening the container 12 or after fully opening the container 12. If the disposable changing mat 26 is included in the disposable diaper changing kit 10, the user can support the child on the disposable changing mat 26 during cleaning and changing of the child. The user then removes the soiled diaper from the child and drops the soiled diaper into the container 12 through the open end of the container 12, which now is functioning as a free-standing receptacle 20 for waste. The user then uses the disposable wipes 24 to clean soiled areas of the child and drops the used disposable wipes 24 into the container 12. Unused disposable wipes 24 could be kept by the user or also disposed of in the container 12. The user places the clean disposable diaper 22 on the child. The user can then remove the child from the disposable changing mat 26 after use and drop the disposable changing mat 26 into the container 12. If the hand sanitizing packet 28 is included in the disposable diaper changing kit 10, the user can clean his or her hands with the sanitizing liquid or gel or sanitizing wipes in the hand sanitizing packet 28, then drop the used hand sanitizing packet 28 and used sanitizing wipes, if any, into the container 12. The user then closes the open end and disposes of the container 12 with the used and soiled items therein, which can be done by folding, scrunching or twisting the top, or sealing with the sealing structure 30.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A disposable diaper changing kit comprising:
a container comprising a base panel and side panels connected thereto, wherein said container is expandable from a collapsed position in which said side panels and said base panel are folded over and an expanded position in which said side panels are transverse to said base panel and together define a free-standing receptacle with an open end to receive used or soiled items dropped therein, wherein said container comprises a tear-off cover to seal said open end of said container prior to use, said tear-off cover being fully separable from a main portion of said container; and
diaper changing materials comprising a disposable diaper and disposable wipes, wherein said diaper changing materials are stored within said container when said container is in the collapsed position.

2. The disposable diaper changing kit of claim 1, wherein said diaper changing materials comprise a disposable changing mat.

3. The disposable diaper changing kit of claim 1, wherein said diaper changing materials comprise a disposable hand sanitizing packet.

4. The disposable diaper changing kit of claim 2, wherein said diaper changing materials comprise a disposable hand sanitizing packet.

5. (canceled)

6. (canceled)

7. The disposable diaper changing kit of claim 4, wherein said side panels have a height substantially greater than a width of said base panel.

8. (canceled)

9. The disposable diaper changing kit of claim 4, wherein said container comprises a sealing structure configured to seal said open end of said container after used or soiled items are dropped into said container.

10. (canceled)

11. The disposable diaper changing kit of claim 4, wherein said base panel and said side panels comprise a semi-rigid material.

12. (canceled)

13. (canceled)

14. (canceled)
